(19) United States
(12) Patent Application Publication
(10) Pub. No.: US 2026/0204405 A1
(43) Pub. Date: Jul. 16, 2026

(54) ID ENHANCEMENT SYSTEM FOR AI-BASED MEDICAL TOOL IDENTIFICATIONS

(71) Applicant: AURIS HEALTH, INC., Santa Clara, CA (US)

(72) Inventors: Mingyi Zheng, San Jose, CA (US); Hedyeh Rafii-Tari, Mountain View, CA (US); Menglong Ye, Mountain View, CA (US)

(73) Assignee: AURIS HEALTH, INC., Santa Clara, CA (US)

(21) Appl. No.: 19/019,076

(22) Filed: Jan. 13, 2025

(51) Int. Cl. G16H 40/60; G06V 10/764; G06V 10/82; G16H 20/40; G16H 40/40

(52) U.S. Cl. CPC G16H 40/60; G16H 20/40; G16H 40/40; G06V 10/764; G06V 10/82; G06V 2201/034

(57) ABSTRACT

A system for enhancing AI-based identifications of tools comprises memory and control circuitry. The memory is configured to store a first set of AI-based identifications of tools used during a medical procedure and supplemental data related to the medical procedure. The control circuitry is configured to group identifications of tools from the first set into timeblocks, the timeblocks including at least a first timeblock; determine that different tool identifications exist in the first timeblock, the different tool identifications including identifications for at least a first type of tool and a second type of tool; and based at least in part on the supplemental data, filter-out at least one of the first type of tool and the second type of tool from the first timeblock.

### BACKGROUND

### Field

The present disclosure relates generally to the medical field, and specifically to postoperative review of medical procedures.

### Description of Related Art

Various medical procedures involve usage of one or more medical tools in association with a defined workflow of phases/activities/steps. Performances of medical procedures may be recorded for postoperative review, which may advantageously provide insight into how to improve future performances of the same medical procedures.

### SUMMARY

Described herein are systems, devices, and methods to determine characteristics of an object such as size of an object located within a subject's body. The characterization may be performed within a lumen based on images captured by an imaging device positioned at a distal end of an endoscope. For example, the object may be a kidney stone located within a ureter and characterization can involve size or shape estimation of the kidney stone. Real-time characterization of the object can help address discrepancies that may arise between preoperative characterization and endoluminal characterization, which may improve safety of medical procedures and expedite performance thereof.

One innovative aspect of the subject matter of this disclosure can be implemented in a system for enhancing identifications of tools. The system includes control circuitry in communication with a memory configured to store a set of identifications of tools used during a medical procedure and supplemental data related to the medical procedure. The control circuitry is configured to group identifications of tools from the set of identifications into timeblocks, where the timeblocks include at least a first timeblock; determine that different tool identifications exist in the first timeblock, where the different tool identifications include identifications for at least a first type of tool and a second type of tool; and based at least in part on the supplemental data, filter out at least one of the first type of tool and the second type of tool from the first timeblock.

Another innovative aspect of the subject matter of this disclosure can be implemented in a system for enhancing identifications of tools. The system includes control circuitry in communication with a memory configured to store a set of identifications of tools used during a medical procedure and supplemental data related to the medical procedure. The control circuitry is configured to access a set of identifications of tools used during a medical procedure and supplemental data related to the medical procedure; group identifications of tools into timeblocks, where the timeblocks include at least a first timeblock; determine that different tool identifications exist in the first timeblock, where the different tool identifications include a plurality of identifications for a first tool type and at least one identification of a background type; and based at least in part on the supplemental data, merge the plurality of identifications of the first tool type over the at least one identification of the background type in the first timeblock.

Another innovative aspect of the subject matter of this disclosure can be implemented in a system for enhancing artificial intelligence (AI) based identifications of tools used during a medical procedure. The system includes one or more processors in communication with at least one computer-readable memory having stored thereon executable instructions, a set of AI-based identifications of tools used during the medical procedure, and supplemental data related to the medical procedure. The one or more processors are configured to execute the instructions to cause the system to determine a time window within which the set of AI-based identifications of tools are identified; determine that different tool identifications exist in the time window, where the different tool identifications include at least: a first tool type, a second tool type, and a modifiable background type; generate a first score for the first tool type and a second score for the second tool type based at least in part on the supplemental data; select the first tool type over the second tool type based on a comparison of the first score and the second score; and update the time window to include the first tool type but exclude the second tool type and the modifiable background type from the time window.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 illustrates an example medical system, in accordance with one or more examples.

FIG. 2 illustrates a schematic view of different components of the medical system of FIG. 1, in accordance with one or more examples.

FIG. 3 illustrates a block diagram of a data flow between an AI-assisted recognition system and an ID enhancer system, in accordance with one or more examples.

FIG. 4 illustrates various medical tools detectable in an image frame of a medical procedure recording and identifiable into a tool class/type, in accordance with one or more examples.

FIG. 5 illustrates an example user interface for playing back procedure videos of the robotic system, in accordance with one or more examples.

FIGS. 6A, 6B, and 6C illustrate a tool identification enhancement process for generating enhanced identification data, in accordance with one or more examples.

FIGS. 7A and 7B illustrate a threshold-based enhancement process for generating enhanced identification data, in accordance with one or more examples.

FIGS. 8A and 8B illustrate a score-based enhancement process for generating enhanced identification data, in accordance with one or more examples.

FIGS. 9A, 9B, and 9C illustrate a navigation-based enhancement process for generating enhanced identification data, in accordance with one or more examples.

### DETAILED DESCRIPTION

The headings provided herein are for convenience only and do not necessarily affect the scope or meaning of disclosure. Although certain preferred embodiments and examples are disclosed below, the subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

Medical procedures are usually carried out under constrained time and resources and streamlined procedure may help avoid complications in the operating room during performance of the procedure and out of the operating room during recovery. It is common to record performances of procedures as a video or a collection of images for postoperative review and analysis.

However, raw recordings can span hours with little to no guidance on identifying segments of interest. For instance, a reviewer trying to compile a report on circumstances likely to result in successful biopsies may need to manually sort through the recording to find segments depicting biopsy needles. Such tedious task would not be the best use of the reviewer's resources and may best be left to an analytical system.

As an example of the analytical system, an artificial intelligence (AI) assisted recognition system can provide computerized indexing (e.g., segmentation, labelling, or otherwise post-processing) of recordings through automatic recognition of medical tools, phases, activities, and/or workflow in the recordings. The AI-assisted recognition system may use one or more machine learning models to detect/predict tool-presence, identify anatomical features, and/or determine tool pose in relation to the anatomical features in the recordings. For example, in a bronchoscopy workflow, the AI-assisted recognition system could determine a biopsy phase based on detection of a biopsy needle near a nodule. As another example, in a ureteroscopy workflow, the AI-assisted recognition system could determine a capture activity based on detection of a basket closing near a kidney stone. In addition to the computerized indexing, the AI-assisted recognition systems may have significant applications in generating statistics of tools usage, procedure summaries, and reports, all important functions for evaluative, training, and/or archiving purposes.

It is noted that the AI-assisted recognition systems may have significant applications intraoperatively as well. The AI-assisted recognition system may, when performing similar functions on a live video stream, provide real-time notifications regarding reminders, operational suggestions, chance of successes, and other recommendations to assist clinical staff and improve outcomes of procedures. As such, the AI-assisted recognition systems have potential to become part of intelligent clinical suites or context-aware decision support systems in enhanced operating rooms (ORs). However, the AI-assisted recognition must first address certain challenges.

Some challenges may arise from limited visual information available in a recording. For example, when a recording is captured by an endoscopic camera, the recording may show only monocular views with no depth information and narrow field of view. Worse, some portions of the images may be obstructed or obscured by various anatomical features (e.g., blocked by tissue) and a tool may not be fully visible in the views. Thus, the AI-assisted system could be prone to detection of false positives/false negatives.

Some other challenges may relate to continuity or persistence reasons. For example, even when the AI-assisted system correctly detects a tool, if the tool is temporarily obscured from the field of view then reappears during an attempt (e.g., a biopsy attempt), the AI-assisted system may identify the tool instances as from separate attempts. It is also possible that the AI-assisted system may, incorrectly recognizing the reappearance of the same tool as a detection of a new tool, perform another tool recognition process and mistakenly assign a different tool class/type. Incorrect tool identifications can lead to complications in subsequently performed phase/activity/workflow identification.

To address challenges like these, identification (ID) enhancement of the present disclosure may be conducted after tool recognitions to (e.g., post-process AI-driven tool-presence detection and identification). The ID enhancement can remove “noisy” identifications. “Noisy” may refer to tool identifications (tool IDs) that are out of time or out of place, of a different tool, false positive, false negative, or otherwise generally incorrect identifications.

The ID enhancement may involve merging timewise-adjacent tool IDs, filtering out tool IDs, or otherwise cleaning up tool IDs. For example, if two different tool IDs occur within a short timeframe as indicated by timestamp data but the timeframe is too short for a tool to be removed and replaced in a subject, then the ID enhancement may merge the two tool IDs into one more probable tool ID. In another example, if one type of tool is unlikely to be used based on the location of the tool in the subject, the ID enhancement may remove the tool ID based on supplemental location data.

### Medical System

Various aspects of the present disclosure described herein may be integrated into a robotically enabled/assisted medical system, including a surgical robotic system (robotic system for short), capable of performing a variety of medical procedures, including both minimally invasive, such as laparoscopy, and non-invasive, such as endoscopy, procedures. Among endoscopy procedures, the robotically enabled medical system may be capable of performing bronchoscopy, ureteroscopy, gastroscopy, etc.

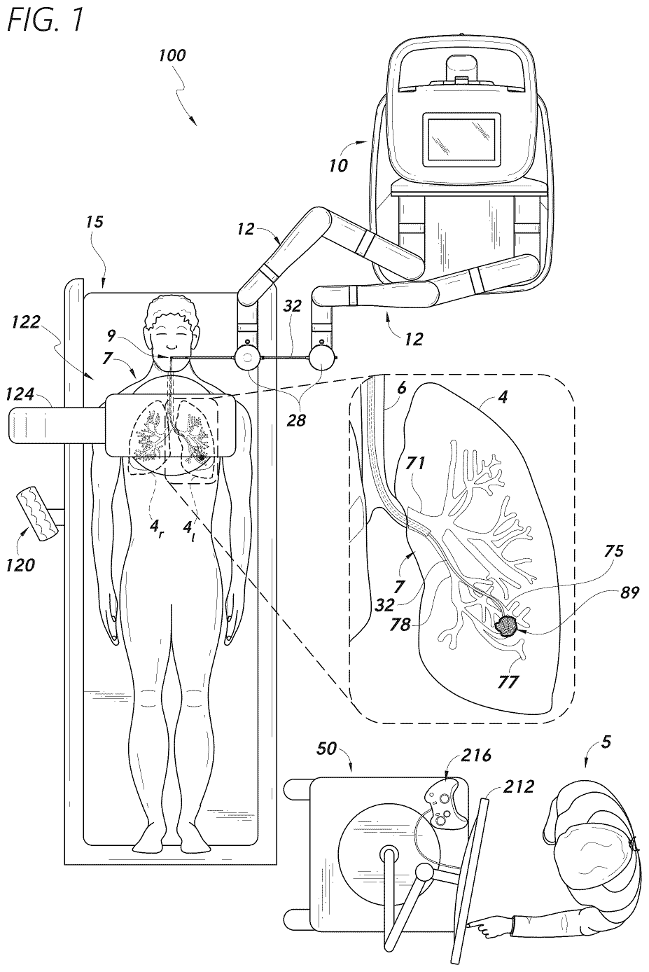

FIG. 1 illustrates an example medical system 100 (also referred to as “surgical medical system 100” or “robotic medical system 100”) in accordance with one or more examples. For example, the medical system 100 can be arranged for diagnostic and/or therapeutic bronchoscopy, as shown. The medical system 100 can include and utilize a robotic system 10, which can be implemented as a robotic cart, for example. Although the medical system 100 is shown as including various cart-based systems/devices, the concepts disclosed herein can be implemented in any type of robotic system/arrangement, such as robotic systems employing rail-based components, table-based robotic end-effectors/manipulators, etc. The robotic system 10 can comprise one or more robotic arms 12 (also referred to as “robotic positioner(s)”) configured to position or otherwise manipulate a medical instrument, such as a medical instrument 32 (e.g., a steerable endoscope or another elongate instrument having a flexible elongated body). For example, the medical instrument 32 can be advanced through a natural orifice access point (e.g., the mouth 9 of a subject 7, positioned on a table 15 in the present example) to deliver diagnostic and/or therapeutic treatment. Although described in the context of a bronchoscopy procedure, the medical system 100 can be implemented for other types of procedures, such as gastro-intestinal (GI) procedures, renal/urological/nephrological procedures, etc. The term “subject” is used herein to refer to live patient as well as any subjects to which the present disclosure may be applicable. For example, the “subject” may refer to subjects including physical anatomic models (e.g., anatomical education model, anatomical model, medical education anatomy model, etc.) used in dry runs, models in computer simulations, or the like that covers non-live patients or test subjects.

With the robotic system 10 properly positioned, the medical instrument 32 can be inserted into the subject 7 robotically, manually, or a combination thereof. In examples, the one or more robotic arms 12 and/or instrument driver(s) 28 thereof can control the medical instrument 32. The instrument driver(s) 28 can be repositionable in space by manipulating the one or more robotic arms 12 into different angles and/or positions.

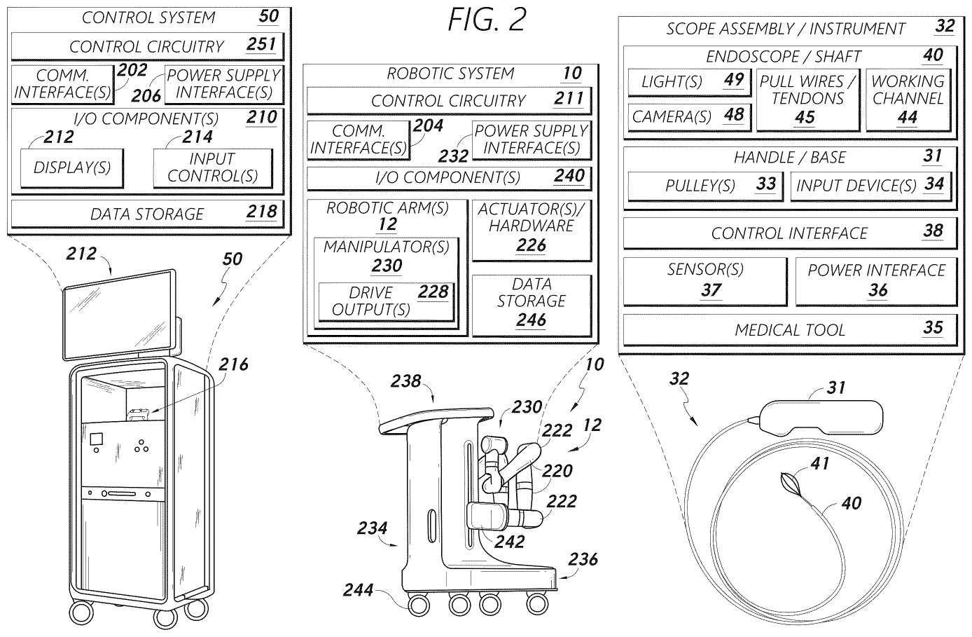

The medical system 100 can also include a control system 50 (also referred to as “control tower” or “mobile tower”), described in detail below with respect to FIG. 2. The control system 50 can include one or more displays 212 to provide/display/present various information related to medical procedures, such as anatomical images. The control system 50 can additionally include one or more control mechanisms, which may be a separate directional input control 216 or a graphical user interface (GUI) presented on the displays 212.

In some examples, the display 212 can be a touch-capable display, as shown, that may present anatomical images and allow selection thereon. Few example anatomical images can include CT images, fluoroscopic images, images of an anatomical map, or the like. With the touch-capable display, an operator 5 reviewing the images may find it convenient to identify targets (e.g., target objects or a target region of interest) within the images using a touch-based selection instead of using the directional input control 216. For example, the operator 5 may select a scope tip and/or a nodule using a touchscreen.

The control system 50 can be communicatively coupled (e.g., via wired and/or wireless connection(s)) to the robotic system 10 to provide support for controls, electronics, fluidics, optics, sensors, and/or power to the robotic system 10. Placing such functionality in the control system 50 can allow for a smaller form factor of the robotic system 10 that may be more easily adjusted and/or re-positioned by an operator 5. Additionally, the division of functionality between the robotic system 10 and the control system 50 can reduce operating room clutter and/or facilitate efficient clinical workflow.

The medical system 100 can include an electromagnetic (EM) field generator 120, which is configured to broadcast/emit an EM field that is detected by EM sensors, such as a sensor associated with the medical instrument 32. The EM field can induce small currents in coils of EM sensors (also referred to as “position sensors”), which can be analyzed to determine a pose (position and/or angle/orientation) of the EM sensors relative to the EM field generator 120. In some examples, the EM sensors may be positioned at a distal end of the medical instrument 32 and a pose of the distal end may be determined in connection with the pose of the EM sensors. Although EM fields and EM sensors are described in many examples herein, position sensing systems and/or sensors can be any type of position sensing systems and/or sensors, such as optical position sensing systems/sensors, image-based position sensing systems/sensors, etc.

The medical system 100 can further include an imaging system 122 (e.g., a fluoroscopic imaging system) configured to generate and/or provide/send image data (also referred to as “image(s)”) to another device/system. For example, the imaging system 122 can generate image data depicting anatomy of the subject 7 and provide the image data to the control system 50, robotic system 10, a network server, a cloud server, and/or another device. The imaging system 122 can comprise an emitter/energy source (e.g., X-ray source, ultrasound source, or the like) and/or detector (e.g., X-ray detector, ultrasound detector, or the like) integrated into a supporting structure (e.g., mounted on a C-shaped arm support 124), which may provide flexibility in positioning around the subject 7 to capture images from various angles without moving the subject 7. Use of the imaging system 122 can provide visualization of internal structures/anatomy, which can be used for a variety of purposes, such as navigation of the medical instrument 32 (e.g., providing images of internal anatomy to the operator 5), localization of the medical instrument 32 (e.g., based on an analysis of image data), etc. In examples, use of the imaging system 122 can enhance the efficacy and/or safety of a medical procedure, such as a bronchoscopy, by providing clear, continuous visual feedback to the operator 5.

In the interest of facilitating descriptions of the present disclosure, FIG. 1 illustrates a respiratory system as an example anatomy. The respiratory system includes the upper respiratory tract, which comprises the nose/nasal cavity, the pharynx (i.e., throat), and the larynx (i.e., voice box). The respiratory system further includes the lower respiratory tract, which comprises the trachea 6, the lungs 4 (4r and 4l), and the various segments of the bronchial tree. The bronchial tree includes primary bronchi 71, which branch off into smaller secondary 78 and tertiary 75 bronchi, and terminate in even smaller tubes called bronchioles 77. Each bronchiole tube is coupled to a cluster of aveoli (not shown). During the inspiration phase of the respiratory cycle, air enters through the mouth and nose and travel down the throat into the trachea 6, into the lungs 4 through the right and left main bronchi 71, into the smaller bronchi airways 78, 75, into the smaller bronchiole tubes 77, and into the alveoli, where oxygen and carbon dioxide exchange takes place.

The bronchial tree is an example luminal network in which robotically-controlled instruments may be navigated and utilized in accordance with the inventive solutions presented here. However, although aspects of the present disclosure are presented in the context of luminal networks including a bronchial network of airways (e.g., lumens, branches) of a subject's lung, some examples of the present disclosure can be implemented in other types of luminal networks, such as renal networks, cardiovascular networks (e.g., arteries and veins), gastrointestinal tracts, urinary tracts, etc.

In some examples, the imaging system 122 can be configured to capture/update/present images of the anatomy intraoperatively using a CBCT imaging system. During CBCT imaging, the subject 7 may be positioned on the table 15 between an X-ray source and detector mounted on the C-shaped arm support 124 where X-ray beams are passed through a target anatomy, and the resulting images are updated intraoperatively. For example, regarding the lungs 4 of the subject, one or more CBCT captured images or a reconstructed 3D model may be presented to the operator 5 on the display 212. While CBCT is described, it will be understood that the present disclosure contemplates any other imaging techniques capable of providing a 3D reconstruction, such as the normal CT imaging technique.

FIG. 2 illustrates example components of the control system 50, robotic system 10, and medical instrument 32, in accordance with one or more examples. The control system 50 can be coupled to the robotic system 10 and operate in cooperation therewith to perform a medical procedure. For example, the control system 50 can include communication interface(s) 202 for communicating with communication interface(s) 204 of the robotic system 10 via a wireless or wired connection (e.g., to control the robotic system 10). Further, in examples, the control system 50 can communicate with the robotic system 10 to receive position/sensor data therefrom relating to the position of sensors associated with an instrument/member controlled by the robotic system 10. In some examples, the control system 50 can communicate with the EM field generator 120 to control generation of an EM field in an area around a subject 7. The control system 50 can further include a power supply interface(s) 206.

The control system 50 can include control circuitry 251 configured to cause one or more components of the medical system 100 to actuate and/or otherwise control any of the various system components, such as carriages, mounts, arms/positioners, medical instruments, imaging devices, position sensing devices, sensor, etc. Further, the control circuitry 251 can be configured to perform other functions, such as cause display of information, process data, receive input, communicate with other components/devices, and/or any other function/operation discussed herein.

The control system 50 can further include one or more input/out (I/O) components 210 configured to assist a physician or others in performing a medical procedure. For example, the one or more I/O components 210 can be configured to receive input and/or provide output to enable a user to control/navigate the medical instrument 32, the robotic system 10, and/or other instruments/devices associated with the medical system 100. The control system 50 can include one or more displays 212 to provide/display/present various information regarding a procedure. For example, the one or more displays 212 can be used to present navigation information including a virtual anatomical model of anatomy with a virtual representation of a medical instrument, image data, and/or other information. The one or more I/O components 210 can include a user input control(s) 214, which can include any type of user input (and/or output) devices or device interfaces, such as a directional input control(s) 216, touch-based input control(s) including gesture-based input control(s), motion-based input control(s), or the like. The user input control(s) 214 may include one or more buttons, keys, joysticks, handheld controllers (e.g., video-game-type controllers), computer mice, trackpads, trackballs, control pads, sensors (e.g., motion sensors or cameras) that capture hand gestures and finger gestures, touchscreens, toggle (e.g., button) inputs, and/or interfaces/connectors therefore. In examples, such input(s) can be used to generate commands for controlling medical instrument(s), robotic arm(s), and/or other components.

The control system 50 can also include data storage 218 configured to store executable instruments (e.g., computer-executable instructions) that are executable by the control circuitry 251 to cause the control circuitry 251 to perform various operations/functionality discussed herein. In examples, two or more of the components of the control system 50 can be electrically and/or communicatively coupled to each other.

The robotic system 10 can include the one or more robotic arms 12 configured to engage with and/or control, for example, the medical instrument 32 and/or other elements/components to perform one or more aspects of a procedure. As shown, each robotic arm 12 can include multiple segments 220 coupled to joints 222, which can provide multiple degrees of movement/freedom. The robotic system 10 can be configured to receive control signals from the control system 50 to perform certain operations, such as to position one or more of the robotic arms 12 in a particular manner, manipulate an instrument, and so on. In response, the robotic system 10 can control, using control circuitry 211 thereof, actuators 226 and/or other components of the robotic system 10 to perform the operations. For example, the control circuitry 211 can control insertion/retraction, articulation, roll, etc. of a shaft of the medical instrument 32 or another instrument by actuating a drive output(s) 228 of a manipulator(s) 230 (e.g., end-effectors) coupled to a base of a robotically-controllable instrument. The drive output(s) 228 can be coupled to a drive input on an associated instrument, such as an instrument base of an instrument that is coupled to the associated robotic arm 12. The robotic system 10 can include one or more power supply interfaces 232.

The robotic system 10 can include a support column 234, a base 236, and/or a console 238. The console 238 can provide one or more I/O components 240, such as a user interface for receiving user input and/or a display screen (or a dual-purpose device, such as a touchscreen) to provide the physician/user with preoperative and/or intraoperative data. The support column 234 can include an arm support 242 (also referred to as “carriage”) for supporting the deployment of the one or more robotic arms 12. The arm support 242 can be configured to vertically translate along the support column 234. Vertical translation of the arm support 242 allows the robotic system 10 to adjust the reach of the robotic arms 12 to meet a variety of table heights, subject sizes, and/or physician preferences. The base 236 can include wheel-shaped casters 244 (also referred to as “wheels 244”) that allow for the robotic system 10 to move around the operating room prior to a procedure. After reaching the appropriate position, the casters 244 can be immobilized using wheel locks to hold the robotic system 10 in place during the procedure.

The joints 222 of each robotic arm 12 can each be independently-controllable and/or provide an independent degree of freedom available for instrument navigation. In some examples, each robotic arm 12 has seven joints, and thus provides seven degrees of freedom, including “redundant” degrees of freedom. Redundant degrees of freedom can allow robotic arms 12 to be controlled to position their respective manipulators 230 at a specific position, orientation, and/or trajectory in space using different linkage positions and joint angles. This allows for the robotic system 10 to position and/or direct a medical instrument from a desired point in space while allowing the physician to move the joints 222 into a clinically advantageous position away from the subject to create greater access, while avoiding collisions.

The one or more manipulators 230 (e.g., end-effectors) can be couplable to an instrument base/handle, which can be attached using a sterile adapter component in some instances. The combination of the manipulator 230 and coupled instrument base, as well as any intervening mechanics or couplings (e.g., sterile adapter), can be referred to as a manipulator assembly, or simply a manipulator. Manipulator/manipulator assemblies can provide power and/or control interfaces. For example, interfaces can include connectors to transfer pneumatic pressure, electrical power, electrical signals, and/or optical signals from the robotic arm 12 to a coupled instrument base. Manipulator/manipulator assemblies can be configured to manipulate medical instruments (e.g., surgical tools/instruments) using techniques including, for example, direct drives, harmonic drives, geared drives, belts and/or pulleys, magnetic drives, and the like.

The robotic system 10 can also include data storage 246 configured to store executable instruments (e.g., computer-executable instructions) that are executable by the control circuitry 211 to cause the control circuitry 211 to perform various operations/functionality discussed herein. In example, two or more of the components of the robotic system 10 can be electrically and/or communicatively coupled to each other.

Data storage (including the data storage 218, data storage 246, and/or other data storage/memory) can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Control circuitry (including the control circuitry 251, control circuitry 211, and/or other control circuitry) can include circuitry embodied in a robotic system, control system/tower, instrument, or any other component/device. Control circuitry can include any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein can further include one or more circuit substrates (e.g., printed circuit boards), conductive traces and vias, and/or mounting pads, connectors, and/or components. Control circuitry can further comprise one or more storage devices, which may be embodied in a single device, a plurality of devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. In examples in which control circuitry comprises a hardware and/or software state machine, analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Functionality described herein can be implemented by the control circuitry 251 of the control system 50 and/or the control circuitry 211 of the robotic system 10, such as by the control circuitry 251, 211 executing executable instructions to cause the control circuitry 251, 211 to perform the functionality.

The scope assembly/medical instrument 32 includes a handle or base 31 coupled to an endoscope shaft. For example, an endoscope 40 (also referred herein as “scope” or “shaft”) can include the elongate shaft including one or more lights 49 and one or more cameras 48 or other imaging devices. The medical instrument 32 can be powered through a power interface 36 and/or controlled through a control interface 38, each or both of which may interface with a robotic arm/component of the robotic system 10. The medical instrument 32 may further comprise one or more sensors 37, such as pressure sensors and/or other force-reading sensors, which may be configured to generate signals indicating forces experienced at/by one or more components of the medical instrument 32.

The medical instrument 32 includes certain mechanisms for causing the scope 40 to articulate/deflect with respect to an axis thereof. For example, the scope 40 may have been associated with a proximal portion thereof, one or more drive inputs 34 associated, and/or integrated with one or more pulleys/spools 33 that are configured to tension/untension pull wires/tendons 45 of the scope 40 to cause articulation of the shaft.

The scope 40 can further include one or more working channels 44, which may be formed inside the elongate shaft and run a length of the scope 40. The working channel 44 may serve for deploying therein a medical tool 35 or a component of the medical instrument 32 (e.g., a lithotripter, a basket 41, forceps, laser, or the like) or for performing irrigation and/or aspiration, out through a distal end of the scope 40, into an operative region surrounding the distal end. The medical instrument 32 may be used in conjunction with a medical tool 35 and include various hardware and control components for the medical tool 35 and, in some instances, include the medical tool 35 as part of the medical instrument 32. For example, as shown, the medical instrument 32 can comprise a basket formed of one or more wire tines but any medical tool 35 are contemplated.

### Tool Identification Enhancement System

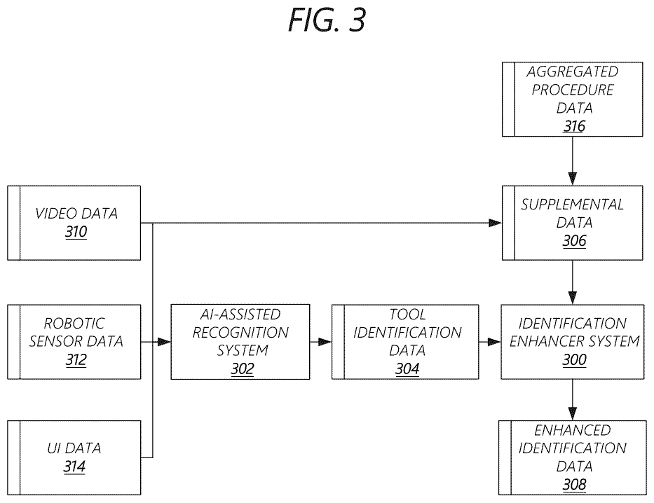

FIG. 3 illustrates a block diagram of the data flow between an AI-assisted recognition system 302 and an ID enhancer system 300, in accordance with one or more embodiments. The AI-assisted recognition system 302 may be part of a robotic system utilized in performing a robotically-assisted medical procedure, such as bronchoscopy, colonoscopy, laparoscopy, gastroscopy, ureteroscopy, or any other procedures, endoscopic or otherwise.

The AI-assisted recognition system 302 can use various data to help control the robotic system (e.g., the medical system 100 of FIG. 1) during the medical procedure as well as to collect procedure data for postoperative analysis. For example, the AI-assisted recognition system 302 can receive various input data including as video data 310 captured by an imaging sensor of the robotic system, robotic sensor data 312 from one or more sensors of the robotic system, user interface (UI) data 314 received from an input device of the robotic system, or the like and collect aggregate procedural data 316.

Video data 310 can include video captured from scopes deployed within a subject, video captured from cameras in the operating room, and/or video captured by cameras of the robotic system. Robotic sensor data 312 can include kinematic data from the robotic system (e.g., using vibration, accelerometer, positioning, and/or gyroscopic sensors), device status, tool location, temperature, pressure, vibration, haptic/tactile features, sound, optical levels or characteristics, load or weight, flow rate (e.g., of target gases and/or liquid), amplitude, phase, and/or orientation of magnetic and electronic fields, constituent concentrations relating to substances in gaseous, liquid, or solid form, and/or the like. UI data 314 can include button presses, menu selections, page selections, gestures, voice commands, and/or the like made by the user and captured by input devices of the robotic system. Patient sensor data, such as heart-rate, oxygenation level, breathing rate and the like may also be used as an input to the AI-assisted recognition system 302.

The aggregated procedure data 316 can include various procedural data including an amount of time taken to perform a procedure or a phase of the procedure, an amount of time a particular tool is used, an amount of time to withdraw a first tool and insert a second tool, an amount of time to attempt an action (e.g., capturing a tissue sample, etc.), a count of tool IDs, and/or similar types of data points. The aggregated procedural data 316 can include data of a single procedure or collective data of multiple same procedures. In some examples, the collective data can be processed to establish norms. The established norms can include minimal amounts, average amounts, median amounts, maximum amounts, or other aggregated statistic or value.

The ID enhancer system 300 can receive tool ID data 304 from the AI-assisted recognition system 302. It is noted that a tool ID, as a term described herein, could refer to the act of identifying a tool as a verb or the resulting identification related to the tool as a noun. Additionally, a tool ID as a noun may refer to either or both of an instance of detected tool-presence (a tool instance) and an identified tool class/type associated with a specific function or structure (e.g., a basket, a needle-like tool, etc.). For example, if the AI-assisted recognition system 302 detects a tool instance from a recording where the tool instance depicts a tool not readily falling into a known tool class/type, the tool-presence detection would be correct (tool ID correctly performed for detecting tool presence) yet the tool class/type may be incomplete or incorrect (tool ID incorrectly associated with a specific tool function). Generally, what is referred by the term tool ID will be apparent based on context of use therein.

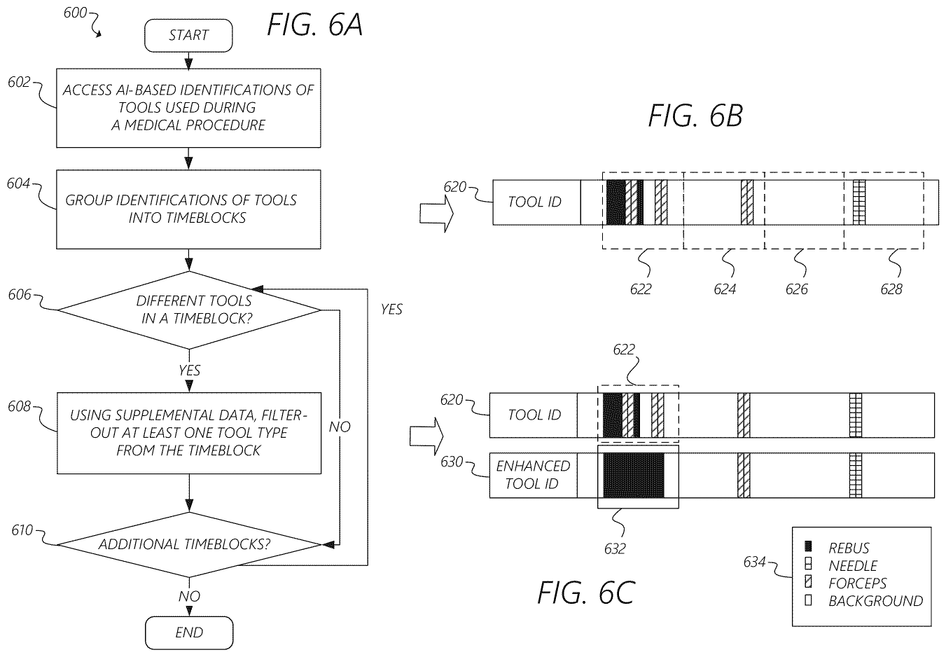

The ID enhancer system 300 can perform various processes on the tool ID data 304 to reduce false positives and otherwise increase the accuracy of the data. The ID enhancer system 300 may use supplemental data 306 to identify these false positives. The supplemental data 306 can include the video data 310, robotic sensor data 312, the UI data 314, as well as aggregated procedure data 316, such as data compiled over multiple procedures (e.g., minimum time it takes to perform an attempt, minimum time it takes to withdraw/introduce a tool, etc.). The ID enhancer system 300 can then process the tool ID data 304, with the help of the supplemental data 306, to remove noise (filter-out incorrect tool ID data 304) or otherwise correct tool ID data 304 that are incorrect. The ID enhancer system 300 can use various algorithms and/or machine learned models to remove noise or correct tool ID data 304. The resulting tool ID data 304 may then provided by the ID enhancer system 300 as enhanced ID data 308. Various enhancement techniques will be described in detail in relation to FIGS. 6-9.

The enhanced ID data 308, cleaned to provide tool ID data 304 without noise, can facilitate automated analysis of phase/activity/workflow recognition. For example, the phases of a medical procedure can become easier to identify with less false positives in the data.

### AI-Based Tool Identification

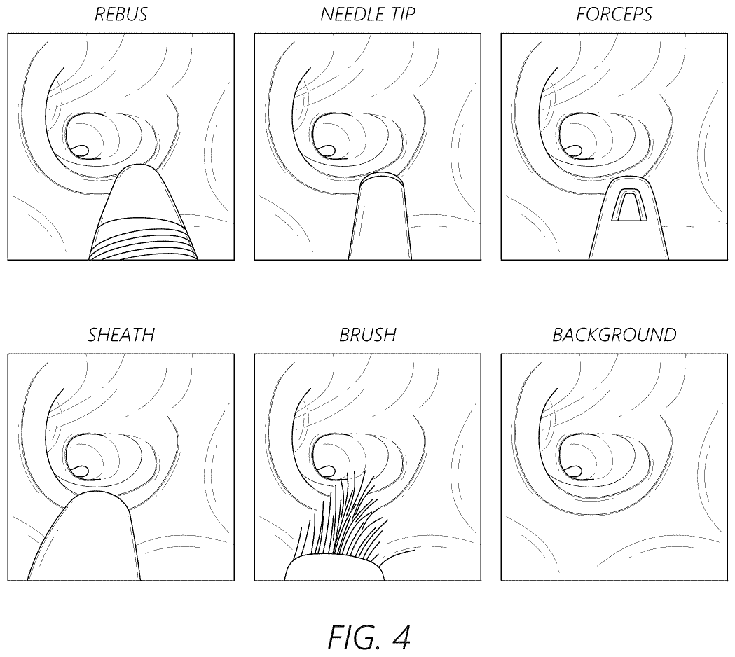

FIG. 4 illustrates various medical tools detectable in an image frame of a medical procedure recording and identifiable into a tool class/type, in accordance with one or more examples. As shown in FIG. 4, some example identifications include radial endobronchial ultrasound (REBUS) tool, needle tip, forceps, sheath, brush, and background. Background refers to an image frame where no tool is shown and only the surroundings are shown in the image frame. Depending on software implementation, the background may be treated as a tool ID that corresponds to no tool or a null tool value (e.g., ‘NULL’, ‘0’, or ‘−1’, where tool IDs of other tools are positive integers) when processing the video. These tool IDs can be made by the AI-assisted recognition system 302 described in FIG. 3.

After identifying the portion of the image associated with the medical tool, AI-based image processing can be performed by the AI-assisted recognition system 302 to identify the medical tool. Additional data (e.g., supplemental data 306 of FIG. 3) from the robotic system performing a medical procedure, such as bronchoscopy, can be used to aid in tool ID. Such additional data can include phase information for the procedure, which can be used to narrow down the possible medical tools based on knowledge of the typical tools used during particular phases of the procedure. For example, during a targeting phase and biopsy phase, the tools likely used are REBUS, needle, brush, and forceps. If the procedure is in those phases, then the possible choices for the tool ID for the tool recorded in a video can be narrowed down to those possibilities.

At the frame level, every frame (image) or a subset of frames (images) of a video can be classified individually as belonging to a tool class/type (e.g., REBUS, needle, forceps, etc.) and/or having a tool ID associated with the tool class/type. Machine learning approaches can be employed to perform such classification and a tool ID as referred herein can involve a tool prediction estimated using a trained machine learning model. In one example, a standard pipeline for achieving this classification may include explicit visual feature extraction on the image, followed by classification with dedicated classifiers that have been previously trained. A classifier may be any algorithm that sorts data into labeled classes, or categories of information. An example is an image recognition classifier to label an image (e.g., “needle,” “brush,” “forceps,” etc.). Classifier algorithms may be trained using labeled data. For instance, an image recognition classifier receives training data that labels images. After sufficient training, the classifier then can receive unlabeled images as inputs and will output classification labels for each image. Classifiers can be decision trees, random forests, or support vector machines. In some examples, models that rely on Convolutional Neural Networks (CNNs) may be used for both image/tool segmentation and tool classification. Example of CNN-based architectures that can be used for this task include ResNet, U-Net, MaskRCNN, and nnU-Net, among others.

Different classes and sub-classes can be defined for this classification process. Classes and sub-classes may range from more general classification of tool class/types to more detailed classifications. For example, more general classifications of images or portions of images can include background, REBUS, needle, and forceps. More detailed or granular classifications (e.g., sub-classes) can include first-party manufacturer sheath, third-party manufacturer sheath, needle tip, forceps tip, brush tip, or the like.

In some examples, tool ID may comprise a tool-presence detection step and an episode recognition step. During image processing, episodes can be identified in the video. In one example, an episode may be a sequence of 8 frames that are labeled based on a tool class identified in the majority (e.g., 4 or more) of the frames across that episode. This may operate using an assumption that an episode, in actuality, only has one class within the 8-frame time window and outlier classifications can be ignored. As will be apparent, other numbers of frames can be used to define an episode.

During the tool-presence detection step and the episode recognition step, a tracked medical tool is categorized into one of several classes/types. In one example, the tool-presence detection step uses six classes and the episode recognition step uses four classes. For example, six classes for tool-presence detection can include REBUS, forceps, brush, needle tip, sheath, and background. In another example, the episode recognition step can use the types of motion or structure identified in the frames of the episode to categorize the episode into one of several classes including REBUS-type, forceps-type, needle-type, and background-type. The classes can include several tools, and a particular tool can be in multiple classes. In one example, the REBUS-type class can include a REBUS and a sheath. The forceps-type class can include forceps and a needle. The needle-type class can include a needle tip, a brush, and a sheath. The background-type can be a catch-all for various images without a medical tool (e.g., passageways, lumen, or any other anatomical sites). As described above, rotational movement can indicate a REBUS tool, dithering can indicate a needle or brush, and a quick pulling motion can indicate forceps.

Different examples of the AI-assisted recognition system 302 may use different types of classifiers or combinations of classifiers. Sequence based models that try to capture the temporal information and sequence of activities in a procedure may be more capable of identifying surgical phase and activity recognition, and can be used at different levels of a procedure (phases/tasks, activities/sub-tasks, workflows, etc.).

Some examples of the AI-assisted recognition system 302 can rely on detecting visual cues in the images, using traditional image processing tools for detecting color, shape, or texture information, and use machine learning and statistical analysis methods such as Hidden Markov Models (HMMs) and Dynamic Time Warping (DTW) to capture the temporal information for classification of phases and activities.

Some examples of the AI-assisted recognition system 302 rely on neural networks and deep learning-based architectures for either or both of capturing the features of the images and incorporating the temporal information, and can be used both for post-processing of entire video sequences as well as for online recognition, while demonstrating improved recognition and classification performance. These examples can use CNNs to extract and capture features of the images, followed by Recurrent Neural Networks (RNNs) such as Long-short term memories (LSTMs), to capture the temporal information and sequential nature of the activities. Temporal Convolutional Networks (TCNs) are another class of more recent architectures that can be used for surgical phase and activity recognition, which can perform more hierarchical predictions and retain memory over the entire procedure (as opposed to LSTMs which retain memory for a limited sequence and process temporal information in a sequential way). Many variations are possible for generation of tool IDs for a video.

### Auto-Indexing Recordings and Intelligent Playback

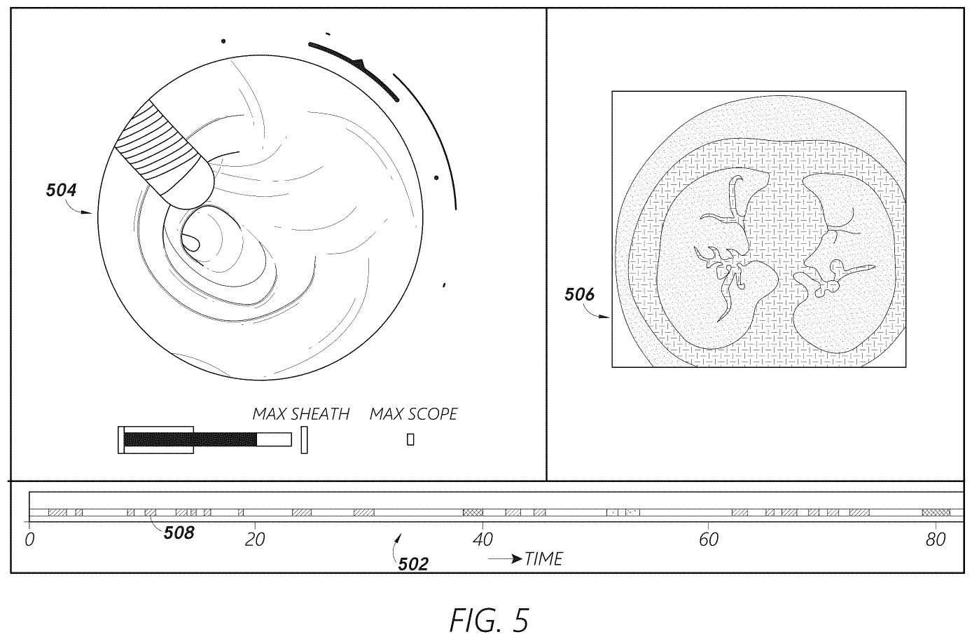

FIG. 5 illustrates an example user interface for playing back procedure videos of the robotic system, in accordance with one or more examples. The user interface may be part of the AI-assisted recognition system 302 that can make identifications for tools used during the medical procedure. The AI-assisted recognition system 302 can also be used to recognize phases, activities, and tool-presence during an operation. In the illustrated example, the user interface includes a seek bar 502, a video view 504, and a procedure overview panel 506.

The seek bar 502 can indicate the detected activities of a procedure with annotations 508 enabling users to directly find the video segment (e.g., one or more consecutive episodes) corresponding to a certain activity or phase, for postoperative analysis. For example, the annotations 508 can index which tool was identified for that time frame. In some examples, the annotations 508 may be colored (e.g., color-indexed) in the seek bar 502 to group segments of the video that are similar, thereby facilitating identification of the similar segments. For example, the segments may be grouped by phase, by activity, and/or by tool class/type. In one example, the annotations 508 are colored to denote a specific tool. Users can then filter to all annotations of one color (e.g., red) to find instances where that tool (e.g., needle) was used. In some examples, different colors can be used to denote the importance/criticality/result/evaluation of a particular activity in the procedure. For instance, red colors (or another visual indicia) may indicate instances where tool was obscured or attempts could be affected.

The seek bar 502 may provide intelligent playback based on the annotations 508. In addition to normal playback for videos including play, pause, rewind, and fast-forward, the intelligent playback can provide controls to jump between annotated segments to streamline review. As alluded, the seek bar 502 may provide filtering functionality which can be configured to show a subset of annotated segments based on tool class/type, phase, activity, importance, or the like.

The video view 504 can present an image frame of a video currently loaded for playback. The procedure overview panel 506 may present a model (e.g., a 3D reconstructed model based on Computerized Tomography) of an anatomical site, tool pose with respect to the model, supplemental data 306 of FIG. 3, or any other operative information pertaining to the procedure recorded.

The AI-assisted recognition system 302 can integrate any operative information into the video view 504 and/or the procedure overview panel 506 or otherwise present the operative information. For example, through application of different computer assisted techniques, such as augmented reality and image overlays, the AI-assisted recognition system 302 may integrate endoscopic ultrasound or other imaging information with the video view 504 and/or the procedure overview panel 506. The integration may be based on the detected tool class/type, estimated tool pose, or identified task/phase of the procedure to provide intelligent guidance including next phase reminders, operational suggestions (e.g., trajectory suggestions, features to avoid, etc.), chance of success, and other recommendations. The intelligent guidance may be provided for postoperative review of captured videos or as intraoperative information for real-time video streams to help outcomes of procedures.

In some examples, the AI-assisted recognition system 302 can incorporate robotic tools with radio-frequency identification (RFID tags), which can allow the system to identify each of the tools (REBUS, Needle, Forceps) through such tags. Tools may be tracked through position sensor information coming from the tools, combined with the kinematic information of the robot. Electromagnetic (EM) and robot kinematic data obtained from the robotic platform can be used together with the machine learning framework for extracting workflow (phase/activity) and skill information, prior to the targeting phase. Other position sensors (e.g., shape sensing) can be used for such purpose as well.

### Tool Identification Enhancement

FIGS. 6A, 6B, and 6C illustrate a tool identification enhancement process 600 for generating enhanced ID data 308, in accordance with one or more examples. FIG. 6A is a flow diagram of the tool ID enhancement process, in accordance with one or more examples. FIGS. 6B and 6C illustrate symbolic representations and example of sub-processes performed in the process 600. For ease of explanation, the tool ID enhancement process 600 is described as being performed by the ID enhancer system 300, referencing elements described in FIG. 3. However, this process may be performed by other systems, such as a robotic system, an AI-based recognition system, or the like. Furthermore, while the following describes one possible sequence to the process, other examples can perform the process in a different order or may include additional steps or may exclude one or more of the steps described below.

At block 602, ID enhancer system 300 can access AI-based identifications of medical tools used during a medical procedure, such as bronchoscopy or other medical procedure. The procedure may be performed or assisted by a robotic system, with a video recorded, offline or online, of the procedure by an imaging device. The video can then be processed by an AI-assisted recognition system 302 to generate tool ID data 304 of medical tools used during the medical procedure.

In some examples, the AI-assisted recognition system 302 and the ID enhancer system 300 are separate devices. The tool ID data 304 can be transferred over a network or via a storage media from the AI-assisted recognition system 302 to the ID enhancer system 300. The tool ID data 304 may be stored in a database, flat file, data file, or other type of data store. In other examples, the AI-assisted recognition system 302 and the ID enhancer system 300 are combined into one system and the tool ID data 304 is stored in a commonly accessible storage media of the combined system.

At block 604, the ID enhancer system 300 can group identifications of medical tools (tool IDs) from a medical procedure into timeblocks. These timeblocks may be a set amount of time (e.g., a time window, a timeframe, a chunk of time, a block of time, etc.) that applies to the entire medical procedure or any portion thereof. In some instances, a timeblock may have a default time duration, such as a minimum time to navigate beyond a trachea. In some instances, a timeblock may have a varying the time duration depending on the phase of the medical procedure. Some phases may take a long time but only use one or two tools (e.g., tool entry into subject), so would benefit from longer timeblocks. Other phases may be shorter but require multiple tools (e.g., tissue sampling at target site), so would benefit from shorter timeblocks. In some instances, a timeblock may have a time duration defined based on supplemental data, such as positional data indicating entry into a right bronchus as provided by an EM sensor, for example.

In some examples, each tool ID includes timing data, such as a time stamp or frame number, referencing the original video recording of the medical procedure. This timing data can be used to group the tool ID into a timeblock corresponding to an amount of time that should only have one type of tool ID. That way, if tool IDs of multiple types of tools are found in the timeblock, it is likely that the timeblock contains an incorrect tool ID. For example, with a timeblock of several video frames up to a few of seconds, it is unlikely that there was sufficient time to deploy a new tool and it can be expected that the timeblock should only have one type of tool. After grouping the tool IDs into timeblocks, each timeblock can then be processed to filter-out incorrect IDs.

FIG. 6B provides an example of a tool ID 620 being divided into, in this particular example, timeblocks 622, 624, 626, 628 with the same amount of time. As alluded, other examples can involve variable sized timeblocks. In the illustrated figure, the first timeblock 622 includes multiple tool IDs of two types of tools, REBUS and forceps as can be seen using the legend 634. The second timeblock 624 and the fourth timeblock 628 each include a tool ID of a single class/type of tool that is, respectively, forceps and a needle. The third timeblock 626 includes identifications of a background image, corresponding to no tool class/type being found.

At block 606, the ID enhancer system 300 can check if different tools types exist in a timeblock. If yes, the process 600 proceeds to block 608. If no, the process 600 proceeds to block 610. Referring back to the example in FIG. 6B, the first timeblock 622 includes two class/types of tools and would proceed to block 608. Meanwhile, the second timeblock 624, third timeblock 626, and fourth timeblock 628 have one tool class/type or only background, so would proceed to block 610.

At block 608, assuming different tool class/types are found in the timeblock, such as in first timeblock 622, the ID enhancer system 300, can remove at least one tool class/type from the timeblock based on supplemental data 306. One particular example of processing a timeblock is shown in FIG. 6C.

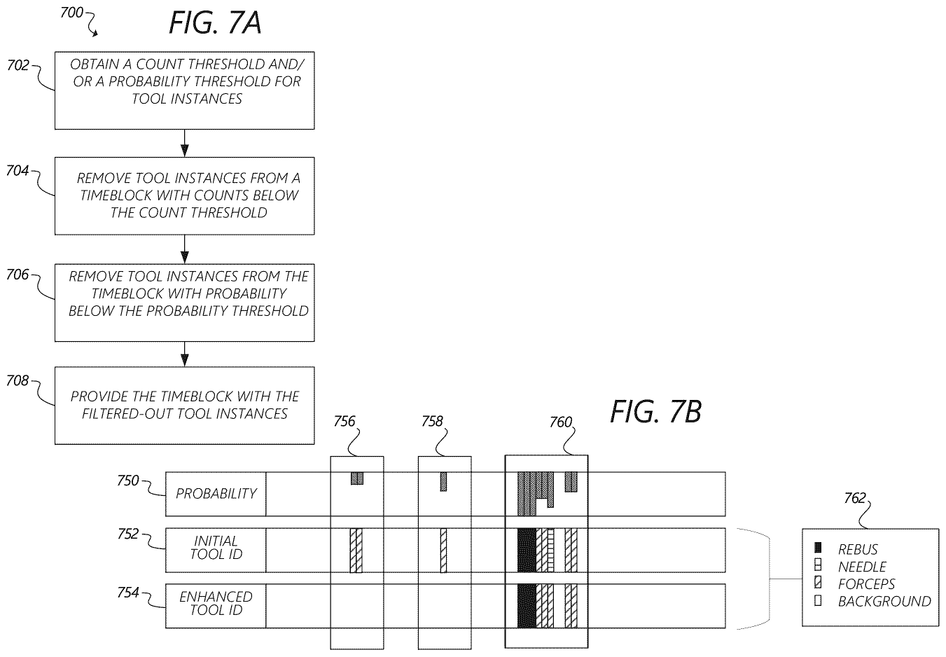

FIG. 6C provides an example of the first timeblock 622 having additional tool IDs filtered out to create an enhanced identification 630 with a filtered-out timeblock 632. In the illustrated figure, the first timeblock 622 includes tool IDs for REBUS, forceps, and background. As will be apparent, these are examples of just some tools that can be identified. Various algorithms can be used to determine which tool ID should be assigned to the first timeblock 622. For example, probabilities, scores, or counts can be used to make the determination as will be described in greater detail in relation to FIGS. 7, 8, and 9. In this example, REBUS is assigned to the filtered-out timeblock 632, with the other indications being removed and replaced with a single indication for REBUS based on supplemental data 306 which could indicate an ultrasound phase of the procedure.

In some examples, optionally, the tool ID enhancement process 600 may compare the order of identified tools against a known order of tools for a procedure. For example, each tool class/type may be expected in certain phases of a procedure in a known order, such as REBUS being the first tool used in a diagnostic phase followed by a needle or forceps in an operational phase in a typical bronchoscopy. The order information of the tool types/class can be used to filter out one or more tool types/classes that are out of order from a timeblock.

At block 610, the ID enhancer system 300 can determine if additional timeblocks exist. If additional timeblocks exist, then the process 600 proceeds back to block 606 so the next timeblock can be processed. If no additional timeblocks exist, the process 600 ends and the enhanced identification 630 can be provided.

### Threshold-Based Enhancement

FIGS. 7A and 7B illustrate a threshold-based enhancement process 700 for generating enhanced ID data 308, in accordance with one or more examples. The threshold-based enhancement process 700 can use information such as count thresholds or probability thresholds to filter out tool IDs. For example, tool IDs that last for only a few frames of video have a low total count compared to the total frames of a timeblock. These tool IDs are likely to be inaccurate and can be removed. In another example, the AI-based identification algorithm may assign probabilities to each tool ID. Tool IDs that do not reach a certain prediction threshold (e.g., 10%, 20%, 30%, 40%, below 50%, etc.) can be removed. Tool IDs that are removed may be completely erased or merged with tool IDs that are nearby in time (e.g., within a number of frames of video, within a few seconds in the video, neighboring tool ID(s), etc.).

FIG. 7A is a flow diagram of the threshold-based enhancement process 700, in accordance with one or more examples. FIG. 7B illustrates a symbolic representation and example of sub-processes performed in the process 700. For ease of explanation, the filtering process 700 is described as being performed by the ID enhancer system 300, referencing elements described in FIG. 3. However, this process may be performed by other systems, such as a robotic system, an AI-based recognition system, or the like. Furthermore, while the following describes one possible sequence to the process, other examples can perform the process in a different order or may include additional steps or may exclude one or more of the steps described below.

The ID enhancer system 300 can filter-out tool IDs deemed incorrect from a timeblock, as described in block 608 of FIG. 6A. The filtering process 700 describes one way of determining which tool IDs to filter out from the timeblock.

At block 702, the ID enhancer system 300 can obtain a count threshold and/or a probability threshold from the supplemental data 306. For example, the supplemental data 306 can include aggregated procedure data 316 that aggregates data from multiple medical procedures of the same type (e.g., bronchoscopy procedures) to generate expected norms. Norms determined from aggregated procedural data 316 can be used to set the count threshold and probability threshold that corresponds to enhance tool ID. Alternatively, the count threshold and/or a probability threshold may be settings that are entered by a user into the ID enhancer system 300 as supplemental data 306 received from a user interface.

At block 704, the ID enhancer system 300 can remove tool instances from a timeblock with counts below the count threshold. An tool instance is an identification of a single tool that may correspond to a variable count of time-units (e.g., an episode described in relation to FIG. 4, seconds, frames, or the like) as detected in the video. Both the length of the tool instance and the count threshold may be specified in the same time-unit for comparison to one another.

In FIG. 7B, the tool ID data 304 includes an initial identification 752 for a medical procedure. Filtering-out the tool instances that are deemed to be inaccurate or have a low-probability of being accurate generates the enhanced identification 754. Three example timeblocks are in FIG. 7B as a first timeblock 756, a second timeblock 758, and a third timeblock 760. As can be seen from the legend 762, the first timeblock 756 has two forceps instances and the second timeblock 758 has one forceps instance. The third timeblock 760 has three REBUS instances, two forceps instances, one needle instance, two background instances, and two forceps instances in that order.

In the example shown in FIG. 7B, the initial identification 752 shows 2 counts of forceps ID in the first timeblock 756 and 1 count of forceps ID in the second timeblock 758. Assuming a count threshold of greater than or equal to 2 counts, the single count of forceps ID in the second timeblock 758 of the initial identification 752 is removed in the enhanced identification 754. The initial identification 752 additionally shows, 1 count of needle ID in the third timeblock 760 that does not satisfy the count threshold of greater than or equal to 2 counts and will be removed. As will be apparent, other count thresholds can be used.

At block 706, the ID enhancer system 300 can remove tool instances from a timeblock with counts below the probability threshold. In the example shown in FIG. 7B, probability values 750 for each instance of a tool ID found in the initial identification 752. As shown in the figure, high confidence identifications are shown in the probability values 750 as a bar that extends along the height of the probability values 750 chart. Low confidence identifications are indicated by shorter bars. Assuming the two lowest probability values are below the probability threshold, the first timeblock 756 would be affected. As shown in the enhanced identification 754, the two instances of forceps ID in the initial identification 752 are removed in the enhanced identification 754. As will be apparent, other probability thresholds can be used, resulting in additional IDs being filtered out.

At block 708, the ID enhancer system 300 can provide the timeblock with the filtered-out tool instances. As shown in FIG. 7B, the enhanced identification 754 shows no tools identified in the first timeblock 756 and the second timeblock 758. In some implementations, the original tool ID is removed. In other implementations, the original tool ID is replaced with a background identification (e.g., a background tool ID) that corresponds to no tool or a null tool value. In yet other implementations, the original tool ID removed may be merged with a neighboring tool ID (e.g., previous or following tool ID). For example, the third timeblock 760 shows the single count of needle ID to be removed, as determined at block 704, is merged with the previously identified forceps tool ID. While the above process 700 includes filtering on using both count threshold and probability threshold, it will be understood that the process 700 can be performed using only one of the thresholds (e.g., only count threshold, only probability threshold).

### Score-Based Enhancement

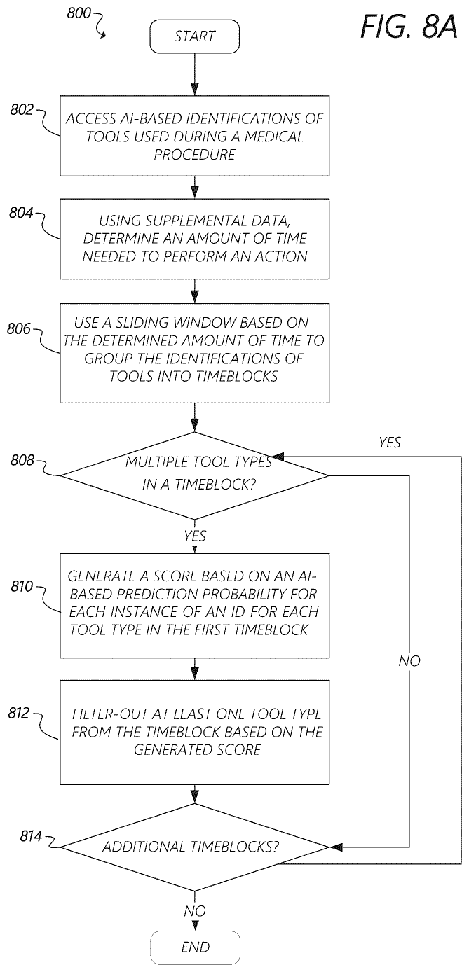

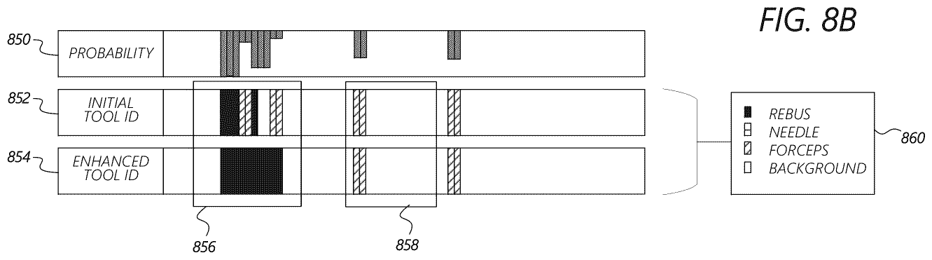

FIGS. 8A and 8B illustrate a score-based enhancement process 800 for generating enhanced ID data 308, in accordance with one or more examples. The score-based enhancement process 800 can use timeblocks (e.g., the timeblocks 622, 624, 626, 628 of FIG. 6B) and probabilities (e.g., the probability values 750 of FIG. 7B) to merge tool IDs that are close together in time. For example, if surrounding video frames are identified as needles with a single frame in the middle identified as a forceps, the forceps ID is likely a mis-identified needle and should be merged with the surrounding tool IDs and converted to a needle ID. Using probability values for tool IDs made by the AI-based identification algorithm, the ID enhancer system 300 can generate a score for each class/type of tool ID found within a certain block of time. The tool ID type with the highest score can be deemed the most likely to be accurate tool and the tool IDs in the timeblock can all be converted to be of that tool class/type.

FIG. 8A is a flow diagram of the score-based enhancement process 800, in accordance with one or more examples. FIG. 8B illustrates a symbolic representation and example of sub-processes performed in the process 800, in accordance with one or more examples. For ease of explanation, the score-based enhancement process 800 is described as being performed by the ID enhancer system 300, referencing elements described in FIG. 1. However, this process may be performed by other systems, such as a robotic system, an AI-based recognition system, or the like. Furthermore, while the following describes one possible sequence to the process, other examples can perform the process in a different order or may include additional steps or may exclude one or more of the steps described below.

At block 802, the ID enhancer system 300 can access AI-based identifications of medical tools used during a medical procedure, such as bronchoscopy or other medical procedure. The procedure may be performed or assisted by a robotic system, with a video recorded of the procedure by the endoscope. The video can then be processed by an AI-assisted recognition system 302 to generate tool ID data 304 of medical tools used during the medical procedure.

In some examples, the AI-assisted recognition system 302 and the ID enhancer system 300 are separate devices. The tool ID data 304 can be transferred over a network or via a storage media from the AI-assisted recognition system 302 to the ID enhancer system 300. The tool ID data 304 may be stored in a database, flat file, data file or other type of data store. In other examples, the AI-assisted recognition system 302 and the ID enhancer system 300 are combined into one system and the tool ID data 304 is stored in a commonly accessible storage media of the combined system.

At block 804, the ID enhancer system 300 can determine an amount of time needed to perform an attempt at an action during the medical procedure using supplemental data 306. An action can include retracting and inserting a tool (e.g., a minimum amount of time required in between attempts), tissue sampling, removal of secretions, blood, foreign object, or diseased tissue, installing a medical device, applying medicine, navigating through an area, or the like. Using the supplemental data 306, the ID enhancer system 300 can determine norms for that action, such as an average time, a maximum time, or a minimal time to perform that action.

The amount of time may be constant or may be variable. Further, some amount of time may be a dynamically determinable variable time. For instance, in endoscopic procedures, the minimum amount of time for retracting and inserting a tool may be determined as a function of the tool insertion depth into a luminal network divided by the maximum insertion/retraction speed of the robotic system. In some examples, the amount of time may change based on the types of actions performed in the phase of the medical procedure being analyzed. In one example, the latter phase of bronchoscopy can involve tissue sampling and can take more time than simply navigating through a small area. The sliding window size may be larger in the latter phase than in an earlier navigation phase.

At block 806, the ID enhancer system 300 can use a sliding window based on the determined amount of time to group the tool IDs into timeblocks, each timeblock having potential to be identified with at most a single identified tool. In some examples, the sliding window may be the minimum amount of time required in between attempts with a tool type and/or changing tool types (e.g., changing medical tools 35 in a working channel 44 of FIG. 2 by removal of a first tool and introduction of a second tool into the working channel 44). As discussed previously, by grouping the tool IDs into timeblocks that are unlikely to be long enough to allow another tool to be utilized, the timeblocks can be reduced to a single tool ID. This makes it easier to process the timeblocks as timeblocks with multiple class/types of tool IDs are likely to include inaccurate tool IDs. In some examples, the timeblocks may partially overlap with one another as the sliding window traverses the recorded tool IDs (e.g., the tool ID data 304 of FIG. 3) or in real-time.

It was previously described that backgrounds (e.g., scene within anatomy captured by an imaging device) may be assigned a default background tool ID, such as ‘0.’ In some implementations, backgrounds having a duration less than the length of the sliding window may be differentiated from the default background tool ID by assigning another tool ID (e.g., a mergeable background tool ID such as, for example, ‘−1.’).

At block 808, the ID enhancer system 300 can determine if there are multiple tool types in a timeblock being processed. In the determination, the mergeable background tool ID (more broadly, a modifiable background tool ID to be contrasted with unmergeable/unmodifiable background tool ID) may be considered as an instrument type as well. If yes, the process 800 proceeds to block 810. If no, the process 800 proceeds to block 814. Referring to the example of FIG. 8B, the first timeblock 856 would lead the process 800 to block 810.

In FIG. 8B, the tool ID data 304 includes an initial identification 852 for a medical procedure and probability values 850 for each instance of a tool ID found in the initial identification 852. As shown in the figure, high confidence identifications are shown in the probability values 850 as a tall bar that extends along the height of the probability values 850 chart. Low confidence identifications are indicated by shorter bars. In the initial identification 852, the first timeblock 856 shows several tool instances for REBUS, forceps, and background. In the first timeblock 856 of the enhanced identification 854, these tool instances are merged into a single merged tool ID for a REBUS tool.

At block 810, the ID enhancer system 300 can generate a score for each identified tool based on the probability values 850 and count of the identified tool instances. Referring to the example of FIG. 8B, the first timeblock 856 includes several tool instances of REBUS and several instances of forceps.

In the first timeblock 856 of the initial identification 852, the identified tool class/types are [REBUS, forceps, REBUS, mergeable background, forceps] as indicated by the legend 860. Assuming each tool ID was associated with corresponding tool instance counts of [90, 60, 30, 60, 60], where counts are based on a time-unit that the tool is identified in the video. For example, the time-unit may be 1 video frame, 8 video frames, a few seconds of time, etc. Thus, the counts for REBUS are [90, 30] (the first and the third counts) and the counts for forceps are [60, 60] (the second and the fifth counts). In this example, assume the probability values 850 for each instance of a tool ID are [REBUS: 0.9, forceps: 0.2, REBUS: 0.8, mergeable background 0.8, forceps: 0.2]. Thus, the probabilities for REBUS are [0.9, 0.8] (the first and the third probability values 850) and for forceps [0.2, 0.2] (the second and the fifth probability values 850). One way to calculate a score for each tool ID is to multiply the probability by the count. For example, the REBUS score would be 105=(90*0.9+30*0.8), while the forceps score would be 24=(60*0.2+60*0.2). In this example, a comparison of scores would show that the REBUS has a higher score (105>24), so REBUS would be assigned as the identified tool for the first timeblock 856 in the enhanced identification 854.

At block 812, the ID enhancer system 300 can filter out at least one tool class/type from the timeblock based on the generated score. In the first timeblock 856, since the REBUS was the highest scoring identified tool, the other tool instances of the forceps are filtered out from the initial identification 852 and merged with the highest scoring identified tool REBUS in the enhanced identification 854. Similarly, the mergeable background instances, that were converted from default background instances due to their durations falling below a certain time duration (e.g., the sliding window), are filtered out and merged into the highest scoring identified tool REBUS. The removal of the mergeable background instances through merging into the highest scoring identified tool operates under the assumption that mergeable background instances for a short amount of time (e.g., a few seconds, less than 10 seconds, etc.) is likely due to an occlusion blocking the tool or the tool temporarily moving out of frame. In this situation, the mergeable background instances should be removed. However, background instances that last for a longer amount of time than the sliding window and remain as default background instances are likely due to the tool being withdrawn so those background instances should be retained. FIG. 8B shows a second timeblock 858 where the default background instances are left unfiltered out from the initial identification 852 and left unmerged in the enhanced identification 854.

The filtering and merging may continue until there remains only a single tool class/type and the default background instances in a timeblock. In some examples, as shown in the first timeblock 856, the filtering can remove a lower-scoring tool type and merge instances of the lower-scoring tool type into a higher-scoring tool type. In some examples, the filtering can merge multiple smaller instances of the same tool type into a larger instance of the same tool type (e.g., four REBUS instances are merged into one REBUS instance for a timeblock) by filtering out mergeable background instances and merging neighboring tool IDs of the same tool type. Many variations are possible.

At block 814, the ID enhancer system 300 can determine if additional timeblocks exist to be processed. If additional timeblocks exist, then the process 800 proceeds back to block 808 so the next timeblock can be processed. If no additional timeblocks exist, the process 800 ends and the enhanced identification can be provided.

### Navigation-Based Enhancement

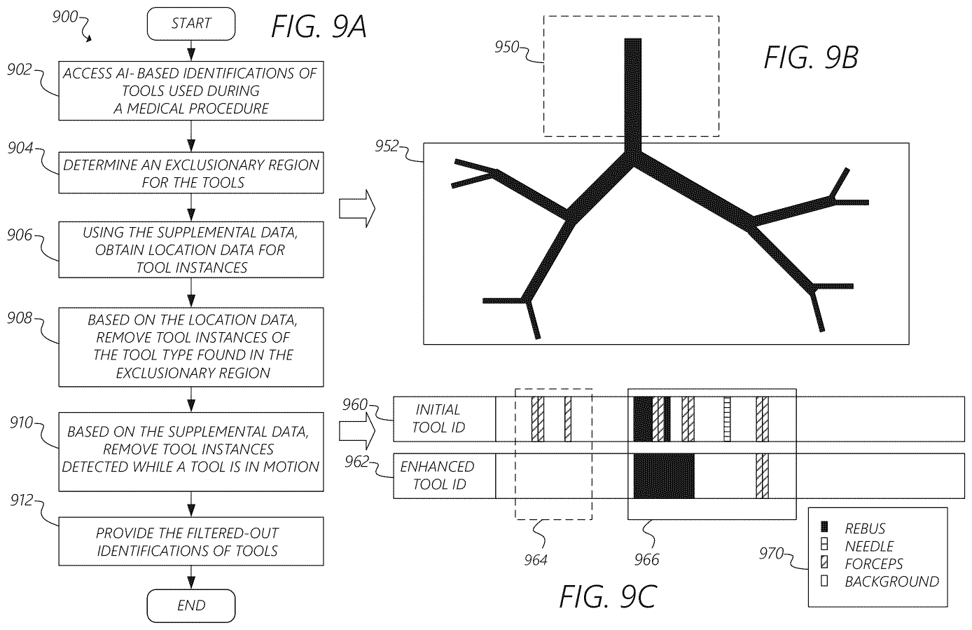

FIGS. 9A, 9B, and 9C illustrate a navigation-based enhancement process 900 for generating enhanced ID data 308, in accordance with one or more examples. The third enhancement process 900 can use tool navigation or tool location data to determine a location in the subject associated with a tool ID. The algorithm may also determine whether the position makes sense for the tool ID based on where particular tools are expected to be used. For example, some tools may not be deployed while a scope is moving towards a target tissue site but only when sampling tissue at the target site. Therefore, tool IDs of that class/type of tool away from the target site are likely to be incorrect as false detections and can be filtered-out by the ID enhancer system 300.

FIG. 9A is a flow diagram of the navigation-based enhancement process 900, in accordance with one or more examples. FIG. 9B illustrates a respiratory system map of a bronchoscopy procedure, in accordance with one or more examples. FIG. 9C illustrates a symbolic representation and example of sub-processes performed in the process 900. For ease of explanation, the navigation-based enhancement process 900 is described as being performed by the ID enhancer system 300, referencing elements described in FIG. 1. However, this process may be performed by other systems, such as a robotic system, an AI-based recognition system, or the like. Furthermore, while the following describes one possible sequence to the process, other examples can perform the process in a different order or may include additional steps or may exclude one or more of the steps described below.

At block 902, the ID enhancer system 300 can access AI-based identifications of tools used during a medical procedure, such as bronchoscopy or other medical procedure. The procedure may be performed or assisted by a robotic system, with a video recorded of the procedure by the endoscope. The video can then be processed by an AI-assisted recognition system 302 to generate tool ID data 304 of medical tools used during the medical procedure.

At block 904, the ID enhancer system 300 can determine an exclusionary region for an tool class/type. In some procedures, certain medical tools are used only in specific locations. For example, during a bronchoscopy operation, an endoscope travels through the respiratory system, starting from the throat and then the trachea. The trachea (e.g., the trachea 6 of FIG. 1) is a tube that connects the larynx in the throat with the bronchi and serves as the main airway for breathing. The trachea splits into two bronchi (e.g., the bronchi 71 of FIG. 1), a left bronchus leading to the left lung and a right bronchus leading to the ring lung. The bronchi further divide into smaller branches called bronchioles (e.g., the bronchioles 77 of FIG. 1), at the end of alveoli. While the endoscope is traveling through the trachea and either the left or right bronchi, no medical tools are likely to be deployed. Thus, the trachea, left bronchus, and/or the right bronchus can be deemed as part of the exclusionary zone. In the example of FIG. 9B, the trachea is marked as the part of the exclusionary zone 950, with the other parts of the respiratory system marked as an active zone 952.

At block 906, the ID enhancer system 300 can obtain location data for tool instances that are part of the AI-based IDs of medical tools. The location data may be obtained using various techniques. For example, the location data may come from tracking sensors used during the medical procedure, estimated based on distance travelled from entry into the subject, or estimated based on time since beginning of the procedure. As shown in the example of FIG. 9C, a first set of tool IDs 964 corresponding to the exclusionary zone 950 are identified, while a second set of tools IDs 966 correspond to the active zone 952 are also identified. The legend 970 identifies the instances of tool class/types shown in FIG. 9C.

At block 908, the ID enhancer system 300 can remove tool instances of the tool class/type(s) found in the exclusionary zone 950, based on the location data. As shown in FIG. 9C, the first set of tool IDs 964 corresponding to the exclusionary zone 950 that are found in the initial identification 960 are removed in the enhanced identification 962.

At block 910, optionally, the ID enhancer system 300 can remove tool instances detected while a medical instrument is in motion. The medical instrument can be an endoscope housing the tools. Whether the medical instrument is in motion may be determined based on location data of the medical instrument over time or robot command data provided by the robotic system. This block 910 is optional for tools that known to be operated only when the medical instrument is stationary.

At block 912, the ID enhancer system 300 can provide the filtered-out IDs of medical tools. In the example of FIG. 9C, the filtered-out results are shown as the enhanced identification 962. The process 900 can then end.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

The term “control circuitry” is used herein according to its broad and ordinary meaning, and can refer to any collection of one or more processors, processing circuitry, processing modules/units, chips, dies (e.g., semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A system for enhancing identifications of tools, the system comprising:
memory configured to store a first set of identifications of tools used during a medical procedure and supplemental data related to the medical procedure; and
control circuitry in communication with the memory, the control circuitry configured to:
group identifications of tools from the first set into timeblocks, the timeblocks including at least a first timeblock;
determine that different tool identifications exist in the first timeblock, the different tool identifications including identifications for at least a first type of tool and a second type of tool; and
based at least in part on the supplemental data, filter-out at least one of the first type of tool and the second type of tool from the first timeblock.

2. The system of claim 1, wherein filtering-out at least one of the first type of tool and the second type of tool from the first timeblock comprises:
generating a first score for the first type of tool based at least in part on an identification probability for each instance of a tool identification for the first type in the first timeblock;
generating a second score for the second type of tool based on an identification probability for each instance of a tool identification for the second type in the first timeblock; and
selecting which of the first type of tool or the second type of tool to filter-out from the first timeblock based on a comparison of the first score and the second score.

3. The system of claim 1, wherein filtering-out at least one of the first type of tool and the second type of tool from the first timeblock comprises:
generating a score for each tool identification in the first timeblock based on an identification probability corresponding to each instance of the tool identification; and
selecting a single tool identification to assign to the first timeblock based at least in part on the generated score.

4. The system of claim 1, wherein the supplemental data comprises an exclusionary region of an anatomy in which a first tool is not expected to be utilized.

5. The system of claim 4, wherein instances of the first tool detected to be located in the exclusionary region are filtered out from the first timeblock.

6. The system of claim 5, wherein the exclusionary region comprises at least one of a trachea, left main bronchi, and right main bronchi.

7. The system of claim 1, wherein grouping identifications of tools into timeblocks comprises:
determining a first time period corresponding to an amount of time to perform an attempt of an action of the medical procedure based at least in part on the supplemental data, wherein the timeblocks have an amount of time that is based on the first time period.

8. The system of claim 7, wherein the first time period corresponds to a minimal amount of time to perform the attempt of the action of the medical procedure.

9. The system of claim 8, wherein grouping identifications of tools into timeblocks comprises using the minimal amount of time as a sliding window to define the timeblocks.

10. A system for enhancing identifications of tools, the system comprising:
memory configured to store a first set of identifications of tools used during a medical procedure and supplemental data related to the medical procedure; and
control circuitry in communication with the memory, the control circuitry configured to:
access a first set of identifications of tools used during a medical procedure and supplemental data related to the medical procedure;
group identifications of tools into timeblocks, the timeblocks including at least a first timeblock;
determine that different tool identifications exist in the first timeblock, the different tool identifications including a plurality of identifications for a first tool type and at least one identification of a background type; and
based at least in part on the supplemental data, merge the plurality of identifications of the first tool type over the at least one identification of the background type in the first timeblock.

11. The system of claim 10, wherein the different tool identifications further include a second tool type and the control circuitry is further configured to:
generate a first score for the first tool type based on an identification probability for each instance of a tool identification for the first tool type in the first timeblock;
generate a second score for the second tool type based on an identification probability for each instance of a tool identification for the second tool type in the first timeblock; and
select which of the first tool type or the second tool type to filter-out from the first timeblock based on a comparison of the first score and the second score.

12. The system of claim 11, wherein the background type reflects no detection of any tool type.

13. The system of claim 10, wherein grouping identifications of tools into timeblocks comprises:
determining a first time period corresponding to an amount of time to perform an attempt of an action of the medical procedure based at least in part on the supplemental data, wherein the timeblocks have an amount of time that is based on the first time period.

14. The system of claim 13, wherein the first time period corresponds to a minimal amount of time to perform the attempt of the action of the medical procedure.

15. The system of claim 14, wherein grouping identifications of tools into timeblocks comprises using the minimal amount of time as a sliding window.

16. The system of claim 13, wherein the control circuitry is further configured to:
determine that the at least one identification of the background type has a duration less than the first time period; and
in response to the determination that the at least one identification of the background type has the duration less than the first time period, determine that the at least one identification of the background type is mergeable with the plurality of identifications for the first tool type in the first timeblock.

17. The system of claim 13, wherein the first time period is based on a minimal amount of time to remove the tool type from a working channel.

18. The system of claim 10, wherein the merging the plurality of identifications for the first tool type over the at least one identification of the background type in the first timeblock comprises replacing the at least one identification of the background type with one or more identifications of the first tool type.

19. The system of claim 10, wherein the identifications of tools include at least one of a sheath, a needle, a REBUS, a forceps, and a brush.

20. A system for enhancing artificial intelligence (AI) based identifications of tools used during a medical procedure, the system comprising:
at least one computer-readable memory having stored thereon executable instructions, a first set of AI-based identifications of tools used during the medical procedure, and supplemental data related to the medical procedure; and
one or more processors in communication with the at least one computer-readable memory and configured to execute the instructions to cause the system to:
determine a time window within which the first set of AI-based identifications of tools are identified;
determine that different tool identifications exist in the time window, the different tool identifications including at least: a first tool type, a second tool type, and a modifiable background type;
generate a first score for the first tool type and a second score for the second tool type based at least in part on the supplemental data;
select the first tool type over the second tool type based on a comparison of the first score and the second score; and
update the time window to include the first tool type but exclude the second tool type and the modifiable background type from the time window.
